(19) United States
(12) Patent Application Publication
(10) Pub. No.: US 2024/0090812 A1
(43) Pub. Date: Mar. 21, 2024

(54) BIOLOGICAL SIGNAL DETECTION ELECTRODE

(71) Applicant: TECHNO-COMMONS INC., Nagano (JP)

(72) Inventor: Kohei HIGUCHI, Himeji-shi, Hyogo (JP)

(21) Appl. No.: 18/267,266

(22) PCT Filed: Dec. 22, 2020
(86) PCT No.: PCT/JP2020/047824

§ 371 Date: Jun. 14, 2023

(51) Int. Cl. A61B 5/257; A61B 5/265; A61B 5/266

(52) U.S. Cl. CPC A61B 5/257; A61B 5/265; A61B 5/266

(57) ABSTRACT

A biological signal detection electrode stable in performance over a long period of time includes an insulating base material having electrical insulation, an electrode terminal buried in the insulating base material such that a signal detection surface is included in a part of a lower surface of the insulating base material, a conductive gel film provided on the lower surface of the insulating base material to cover the signal detection surface, an extraction electrode which is connected to the electrode terminal and has a terminal unit exposed on an upper surface of the insulating base material, and a magnetic body to be a counterpart of the conductive magnet disposed below the terminal unit in the insulating base material. The magnetic body is given only the function of mechanical connection, and the electrode terminal is formed of a corrosion-resistant material, for example Ag—AgCl, not corroded by the conductive gel film.

### TECHNICAL FIELD

The present invention relates to a biological signal detection electrode for detecting a biological signal such as an electrocardiographic signal, a myoelectric signal, and an electroencephalogram.

### BACKGROUND ART

This type of biological signal detection electrode (hereinafter sometimes simply referred to as “detection electrode”) is in the form of a bandage and is also called a patch, and is used by being adhered to a skin surface of a living body (human body). An example thereof will be described with reference to FIG. 2 (as similar examples, refer to Patent Literatures 1 and 2).

FIG. 2 shows a detection electrode 100 and a sensor substrate 200 detachably attached to the detection electrode 100 in a separated state. The sensor substrate 200 includes a pair of conductive magnets 210 as detachable connectors for the detection electrode 100, and transmits the biological signal detected by the detection electrode 100 to a biological signal measuring device (not shown) or the like by wire or wirelessly.

The detection electrode 100 includes an insulating base material 110 that is electrically insulating and made of, for example, a silicone tape material. A pair of electrode terminals 120 are provided on the insulating base material 110 at a predetermined interval. In this conventional example, the electrode terminal 120 is made of an Au-plated iron plate and is shaped like a dish into which the conductive magnet 210 of the sensor substrate 200 is fitted.

The electrode terminals 120 are provided through the insulating base material 110 to contact with the skin surface of the living body, but a conductive gel film 130 made of an electrolyte is provided on a signal detection surface side (lower surface side in FIG. 2) of each electrode terminal 120.

The conductive gel film 130 has adhesiveness and is composed of, for example, a hydrogel body having an acrylic resin crosslinked body as a resin skeleton and containing water, a polyhydric alcohol, and an electrolyte neutral salt (NaCl) uniformly, which enables ohmic connection between the living body and the electrode terminal 120 and is used to reduce the impedance with the skin surface.

In use, after adhering the detection electrode 100 to the living body, the sensor substrate 200 is attached to the detection electrode 100 by magnetically attracting the conductive magnets 210 to the electrode terminals 120. As a result, the biological signal (for example, the electrocardiographic signal) detected by the electrode terminals 120 and 120 is sucked up to the sensor substrate 200 via the conductive magnets 210 and 210, and transmitted wirelessly or by wire to a biological signal measuring device (not shown) or the like. The reason why the detection electrode 100 and the sensor substrate 200 are separated is that the detection electrode 100 is disposable.

### CITATION LIST

### Patent Literature

[PTL 1] JP-UM-A-H5-15907
[PTL 2] JP-A-2008-200365

### SUMMARY OF INVENTION

### Technical Problem

The problem is that the electrolyte used for the conductive gel film 130 is extremely corrosive, so that the Au-plated iron plate of the electrode terminal 120 and the conductive magnet 210 corrode in a short period of time, which deteriorates the electrical characteristics of the biological signal.

Accordingly, an object of the present invention is to provide a biological signal detection electrode that is stable in performance over a long period of time and highly reliable even though a conductive gel film is used.

### Solution to Problem

In order to solve the above problem, the present invention provides a biological signal detection electrode used by being adhered to a skin surface of a living body, including
an insulating base material having electrical insulation, an electrode terminal buried in the insulating base material such that a signal detection surface is included in a part of a lower surface on a side in contact with the skin surface of the insulating base material, a conductive gel film provided on the lower surface of the insulating base material to cover the signal detection surface, an extraction electrode which is connected to the electrode terminal and has a terminal unit exposed on an upper surface of the insulating base material, and a magnetic body disposed below the terminal unit in the insulating base material.

In the present invention, the electrode terminal is made of a corrosion-resistant material that is not corroded by the conductive gel film.

In the present invention, the corrosion-resistant material is selected from Ag—AgCl, Ag paste, AU, Pt, or carbon.

In the present invention, the extraction electrode is preferably made of Ag paste.

In the present invention, the electrode terminal is more preferably made of Ag paste in the same manner as the extraction electrode.

### Advantageous Effects of Invention

According to the present invention, it is possible to provide a biological signal detection electrode that is stable in performance over a long period of time and highly reliable even though a conductive gel film is used.

### BRIEF DESCRIPTION OF DRAWINGS

FIG. 1 is a cross-sectional view showing a main part of a biological signal detection electrode according to the present invention.

FIG. 2 is a cross-sectional view showing a configuration of a conventional example of a biological signal detection electrode.

### DESCRIPTION OF EMBODIMENTS

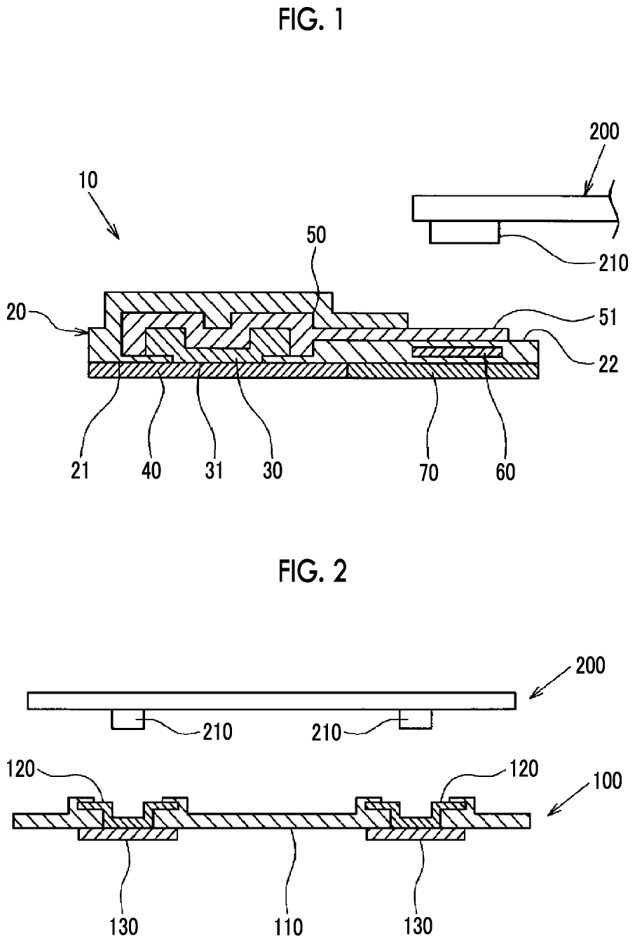

Next, an embodiment of the present invention will be described with reference to FIG. 1, but the present invention is not limited to this.

Referring to FIG. 1, the biological signal detection electrode (detection electrode) of the present invention also has a pair of electrode terminals, but only the electrode terminal on one end side (for example, left side) is shown in FIG. 1, and the electrode terminal on the other end side, which is not shown in FIG. 1, has the same structure as the electrode terminal on one end side. In addition, only one side of the sensor substrate 200 is shown. The sensor substrate 200 may be the same as the sensor substrate previously described in FIG. 2.

The detection electrode 10 (biological signal detection electrode) according to this embodiment includes, as a basic configuration, an insulating base material 20 having electrical insulation, an electrode terminal 30, a conductive gel film 40, an extraction electrode 50, and a magnetic body 60.

In this embodiment, the insulating base material 20 is made of a silicone resin tape material, but is not limited to a silicone resin as long as the resin has flexibility to easily deform depending on the skin surface of a living body. Moreover, the insulating base material 20 is not limited to a strip shape, and may be circular, elliptical, or the like.

The electrode terminal 30 is buried in the insulating base material 20, and in which the electrode terminal 30 is buried in the insulating base material 20 such that a signal detection surface 31, which is the lower surface of the electrode terminal 30, is included in a part of a lower surface 21 on a side in contact with the living body skin surface of the insulating base material 20. That is, the signal detection surface 31 of the electrode terminal 30 is exposed on the lower surface 21 of the insulating base material 20.

The conductive gel film 40 is provided on the lower surface 21 of the insulating base material 20 to cover the signal detection surface 31 of the electrode terminal 30. As in the case of the conductive gel film 130 described in FIG. 2 above, the material of the conductive gel film 40 may be, for example, a hydrogel body having an acrylic resin crosslinked body as a resin skeleton and containing water, a polyhydric alcohol and an electrolyte neutral salt (NaCl) uniformly. This type of hydrogel body includes, for example, “ST-gel” (trade name) manufactured by Sekisui Kasei Co., Ltd.

With such a conductive gel film 40, ohmic connection between the living body and the electrode terminal 30 is enabled and the impedance with the skin surface can be reduced. Although the conductive gel film 40 has adhesiveness, a pressure sensitive adhesive layer 70 may be provided on a portion other than the conductive gel film 40 in order to increase the adhesiveness to the skin surface.

The extraction electrode 50 includes a terminal unit 51 extending laterally from a portion formed to cover an upper surface and side surfaces of the electrode terminal 30 and exposed on an upper surface 22 of the insulating base material 20. A conductive magnet 210 of the sensor substrate 200 contacts the terminal unit 51.

The magnetic body 60 is buried in the insulating base material 20 below the terminal unit 51 as a magnetic attraction counterpart of the conductive magnet 210. In this embodiment, an iron plate is used as the magnetic body 60.

According to this, when the sensor substrate 200 is brought close to the detection electrode 10, the conductive magnet 210 is guided by the magnetic body 60 and contacts with the terminal unit 51, so that the detection electrode 10 and the sensor substrate 200 are connected (contacted) both electrically and mechanically. The magnetic body 60 is not involved in electrical connection but takes only the function of mechanical connection (joint).

In the present invention, the electrode terminal 30 is made of a corrosion-resistant material that is not corroded by the conductive gel film 40. This type of corrosion-resistant material includes Ag—AgCl, Ag paste, AU, Pt, or carbon, but Ag—AgCl (silver-silver chloride) is preferred.

Ag paste is preferably adopted for the extraction electrode 50, but Ag paste may form an entirety in which the electrode terminal 30 and the extraction electrode 50 are integrated.

In any case, according to the present invention, a biological signal detection electrode that is stable in performance over a long period of time and highly reliable without corrosion caused by the conductive gel film 40 even though the conductive gel film 40 is used is provided.

### REFERENCE SIGNS LIST

10: biological signal detection electrode (detection electrode)
20: insulating base material
30: electrode terminal
40: conductive gel film
50: extraction electrode
51: terminal unit
60: magnetic body
70: pressure sensitive adhesive layer

1. A biological signal detection electrode used by being adhered to a skin surface of a living body, comprising:
an insulating base material having electrical insulation;
an electrode terminal buried in the insulating base material such that a signal detection surface is included in a part of a lower surface on a side in contact with the skin surface of the insulating base material;
a conductive gel film provided on the lower surface of the insulating base material to cover the signal detection surface;
an extraction electrode which is connected to the electrode terminal and has a terminal unit exposed on an upper surface of the insulating base material; and
a magnetic body disposed below the terminal unit in the insulating base material.

2. The biological signal detection electrode according to claim 1, wherein the electrode terminal is made of a corrosion-resistant material that is not corroded by the conductive gel film.

3. The biological signal detection electrode according to claim 2, wherein the corrosion-resistant material is selected from Ag—AgCl, Ag paste, AU, Pt, or carbon.

4. The biological signal detection electrode according to claim 1, wherein the extraction electrode is made of Ag paste.

5. The biological signal detection electrode according to claim 4, wherein the electrode terminal is made of Ag paste in the same manner as the extraction electrode.
